(19) United States
(12) Patent Application Publication
(10) Pub. No.: US 2018/0299418 A1
(43) Pub. Date: Oct. 18, 2018

(54) DEVICE AND METHOD FOR CAPTURING AND RECORDING FINE PARTICLES AND/OR THE DENSITY OF NOX GASES IN THE AIR

(71) Applicants: Eric POINCELET, Paris (FR); Philippe POINCELET, Bourg-la-Reine (FR); PLANETWATCH24, Paris (FR)

(72) Inventors: Eric POINCELET, Paris (FR); Philippe POINCELET, Bourg-la-Reine (FR)

(21) Appl. No.: 15/768,753

(22) PCT Filed: Oct. 14, 2016
(86) PCT No.: PCT/FR2016/000166

§ 371 Date: Apr. 16, 2018

(30) Foreign Application Priority Data
Oct. 16, 2015 (FR) 1502183

(51) Int. Cl. G01N 33/00; G01N 15/02; G01N 15/06

(52) U.S. Cl. CPC G01N 33/0032; G01N 15/0205; G01N 2015/0046; G01N 15/0606; G01N 33/0037

(57) ABSTRACT

The invention concerns an on-board device and a mobile method for capturing and recording the fine particles and/or the density of NOx gases in the air for the purpose of dynamically using the gathered data. The device (1) comprises at least one sensor (3) for sensing particles and/or the density of the NOx gases in the air, capable of counting a number of particles or dust particles present in the air, having a dimension ranging between defined values in a volume of air, and a means (7,19) for displaying and/or recording said number and/or density correlated to a determined measurement location of the sensor, the device further being configured to be installed on and to function on a moving vehicle (2) and to be initialised at certain points on the map, or indeed recalibrated, in regular reference to fixed standardised sensors.

The invention relates to a device and method for capturing and recording fine particles and/or the density of NOx gases in the air, in particular for the acquisition and recording of fine particles and/or of the density of NOx gases in the air, notably under mobile conditions and in various places of a city.

It is known that vehicles with heat engines and notably diesel engines, which are numerous in cities, are responsible for much of the pollution of these cities by the discharges of gaseous pollutants from their heat engines and the dust that is emitted, notably fine to very fine particles, and the dust resulting from braking and running of the vehicles. These polluting discharges and dusts harm the health of people exposed to them, notably very fine particles with diameter below 2.5 micrometres, called PM2.5, which enter the respiratory tree and may even reach the bloodstream, thus contributing to long-term health problems, which may be reflected in cardiovascular complications or the development of various forms of cancer.

It is in particular useful for the individual to know the level of these particles in places exposed to a high density of fine particles in order to modulate or adapt his activity (movements and physical activity in particular) as a function of this density and thus preserve his health.

Moreover, for people with delicate bronchi (notably liable to asthma), it is useful to know, in parallel with the particles, the harmful and directly aggressive gases present in the air, notably gaseous nitrogen oxides called NOx, and particularly in the form of nitric acid NO₃.

The invention has the aim of providing a partial or complete remedy for the identification, traceability and finally the resolution of these ills.

A device is proposed according to the invention for capturing and recording fine particles and/or the density of NOx gases in the air, characterized in that it comprises at least one sensor of particles and/or of the density of NOx gases in the air, able to count the number of dust particles or other particles present in the air, of a size between defined values in a volume of air, and/or the density of NOx gases and a means for displaying and/or recording said number and/or density correlated to a particular measurement location of the sensor, the device being configured to be mounted and to function onboard a vehicle (including any mobile support) and in which said sensor may be initialized at certain points of the map, or even recalibrated, with reference to fixed standardized sensors.

The device may also comprise, as mentioned above, a sensor of NOx gases able to measure the density of NOx gases in the air and a means for displaying and/or recording said density correlated to a particular measurement location of the sensor.

Said numbers of particles and density, alone or together, may then be measured, displayed and recorded (in databases) correlated to a particular measurement location, notably a measurement circuit of the vehicle.

Said values are in particular below 2.5 micrometres and correspond to the particles most harmful to human health.

The volume of air considered is the cubic metre. Thus, the number of fine particles per cubic metre of air is recorded.

Said displaying and/or recording means may comprise a simple screen for displaying said number and/or said density, or a device for acquisition of said number and/or said density, on a recording medium, such as a paper printer or a data file.

Said displaying or recording is advantageously carried out on a medium corresponding to a geographical map of said specified measurement location. It is thus possible to determine, continuously or discontinuously, while the vehicle is travelling, a high-definition mapping of the density of fine particles and/or of said density of NOx gases, in the air of said location. This information may be transmitted, in real time or delayed, to at least one database, which may be used via various applications, notably relating to management of the movements and activities of people in the zone assessed with a view to preserving their health on an individual basis.

Said discontinuous determination of the capture of particles and/or of said density of NOx gases may be established at particular measurement points of said location, for example sensitive places of a given geographical zone of the location, each point being allocated measurement of the number of particles and/or of the density of NOx gases, for example the number of fine particles per cubic metre.

Measurement may be effected when the vehicle stops, or while it is moving. Stopping may be delayed according to a specified period to allow re-initialization, or even recalibration, of the sensor at each measurement point, for example by means of a fixed boundary for fine measurement in a stabilized environment, therefore making the measurement at each point reliable without the sensor being affected by the measurement air at the preceding point. Measurement of the particles and/or of the density of NOx gases according to said period is carried out on a measurement circuit, from the vehicle, in said location.

Travel is also possible at a moderate speed in the measurement circuit depending on the capacity for re-initialization of the sensor, for example under 30 km/hour allowing the sensor to perform a measurement “on the fly” (while moving) and with an acceptable margin of error of measurement of the air surrounding the vehicle.

The re-initialization or recalibration of the sensor based on fixed boundaries makes it possible to provide threshold values and therefore guarantee the measured values according to a margin of error below 0.2 or up to max. 0.4.

The particle sensor is preferably an optical dust sensor, for example of the “GP2Y1010AU0F Sharp” type designed to detect particles in the air and thus estimate air quality. Such a sensor, of the type that detects the amount of light passing through a volume of air, makes it possible to display an instantaneous value of the particles in said range of specified values, notably below 2.5 micrometres.

The sensor of NOx gases may be an optical sensor measuring a wavelength spectrum corresponding to the gases in the air.

A sensor of this kind may be supplied with energy by an electric power source of the vehicle, or even by a battery that is recharged from the vehicle.

Said sensor is advantageously mounted on the vehicle in a favourable position for receiving, preferably instantaneously, air to be measured for capturing the particles and/or said density of NOx gases in the air of the measurement point. Said sensor is also configured to minimize the effect of air movement, the wind and/or the vehicle's speed.

The (random) movement of air on the sensor may be minimized by fitting it with a peripheral protective skirt configured to allow air to pass through while moving, for example provided with a grille through which air passes.

Furthermore, the residual air of a given measurement point of the sensor is advantageously evacuated by a device for evacuating the residual air from the sensor, for example a device for blowing neutral air into the sensor, before each new measurement. Said device for evacuating residual air is mounted on the sensor.

Cleaning of the sensor is advantageously performed with ultrasound. This regular, integrated cleaning of the sensor has notably made it possible to perform accurate and reliable measurements in on-board, mobile conditions, which the prior art had not previously allowed, hence limiting the measurements of pollution to the stage of fixed stations, which are very inadequate for accurately measuring the exposure of individuals, notably at breathing height.

The sensor may be mounted on a mast of the vehicle, or a moving, telescopic arm, adjustable for orientation and/or height, notably allowing measurement at variable height, for example on a line of vertical projection from the measurement point, in order to supplement, on a moving vertical axis, the main measurements performed at breathing height.

Measurement may thus be performed at several heights for a given point; it may also be performed in an environment of the given point, at a distance close to the point or to the vertical from the point, for example by orienting the mast and the sensor at a given measurement height.

It is thus possible to determine the density of particles and/or of gases at push-chair height, at the height of a pedestrian on a pavement, at ground floor level or on a higher floor of a residential building, or even inside the building.

It is also possible henceforth to determine the origin and the impact of a source of pollution, for example a chimney or an outlet for discharge of air from for example underground public transport, such as the underground railway or the metropolitan and regional rail system in its immediate environment.

Mounting on a vehicle that does not emit particles (non-polluting, for example with an electric motor) avoids influencing the measurement, in contrast to a vehicle with a heat engine. In the latter case, it is a question of isolating the sensor from the source of fumes from the engine, notably by increasing its distance from the latter, taking into account the wind and the movement of the vehicle relative to the wind, etc.

The device is advantageously coupled to a so-called GPS (Global Positioning System) device.

Thus, the device may be configured to combine, with a GPS geolocation map, localized point display, notably in real time and dynamic, of the density of particles and/or gases, at one or more measurement heights. The map is then supplemented with points of the values of said number of particles and/or of the density of NOx gases.

The recording of values may be sequenced at a specified duration between two consecutive measurement points, for given forward travel of the vehicle corresponding to a given distance travelled so as to obtain a regular point display of said values of particles and/or of the density of NOx gases on the vehicle's circuit.

The sensor may moreover be initialized, or even recalibrated, at certain fixed points of the map, with reference to standardized sensors, for example point and static sensors of PM 2.5 particles of certified environmental monitoring organizations but also of proprietary fixed sensors installed to supplement the mobile network.

The device may comprise a box preferably mounted on the vehicle's dashboard, connected to said GPS device and to the sensor, said box being configured to display and/or record the data from the GPS device and the data from the sensor at each measured point.

Optionally, other data on air quality, such as humidity, haze, electromagnetic waves, noise level in decibels etc., may be displayed and/or recorded.

Preferably, acquisition of the data is stored in memory to be recorded later, notably to constitute a dynamic database.

The box may comprise a screen for displaying the data, notably displaying a high-definition geographical map of the measurement location supplemented with points of said values of the number of particles and/or density of NOx gases, which is convenient for directly monitoring the development of the measurements in space and time.

Advantageously, said device is mounted permanently on the vehicle, for example on the air supply unit of the engine or passenger compartment (secure, vandal-proof installation) but it may have quickly detachable mounting on the vehicle in a compact form (without a measurement mast or arm). Said sensor may be mounted on the windscreen (in an unobtrusive position), the roof or the bonnet of the vehicle, or any other part of the body or external element such as a rear-view mirror able to accommodate said sensor in good capture conditions.

The invention also relates to a method for measuring and displaying and/or recording particles and/or the density of NOx gases in the air by means of at least one sensor of particles and/or of the density of NOx gases in the air, able to count the number of particles of a size between defined values in a volume of air and/or the density of NOx gases, and by means of a device for displaying and/or recording said number and/or density correlated to a defined location, the whole moreover being configured to be mounted and to operate onboard a vehicle (including any mobile support, or even a drone), said method comprising acquisition of said number and/or density at points of a measurement circuit onboard said vehicle.

These features and advantages of the invention and others will become clearer from the following description of embodiments of the invention, referring to the appended drawing, in which:

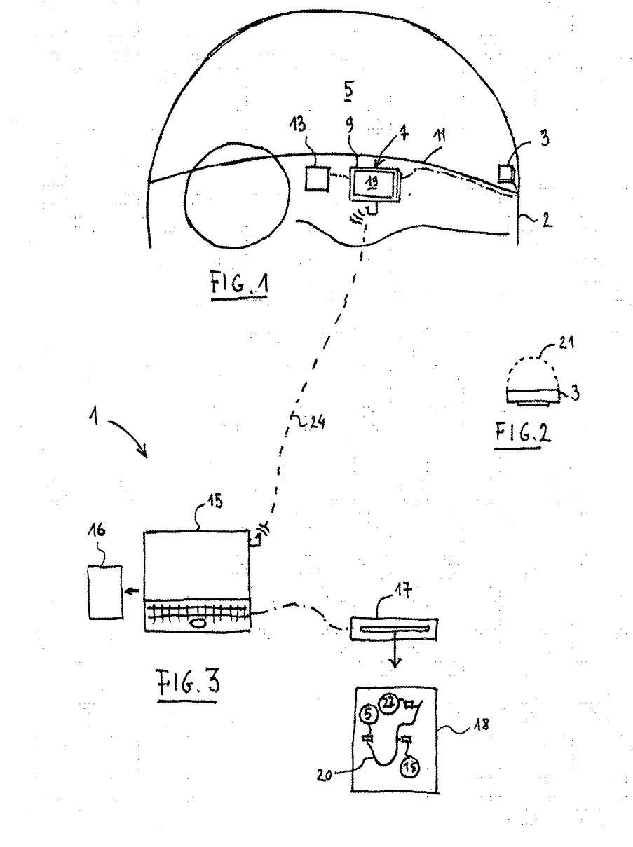

FIG. 1 is a schematic front view of a device for capturing and recording fine particles in the air, mounted on a motor vehicle, viewed from the passenger compartment of the latter, according to one embodiment of the invention,

FIG. 2 is a schematic front view of a particle sensor of said device, and

FIG. 3 shows a device for recording the data from the device, arranged at a distance from the latter and a printed geographical map of absolute or relative density of fine particles in the air in a given geographical zone, obtained using the device.

In the following description, identical numerical references are used for denoting similar or identical elements.

Referring to the drawing, FIG. 1 shows a device 1 for capturing and recording fine particles in the air according to one embodiment of the invention, mounted on a vehicle 2. Here, this device comprises at least one sensor 3 of particles in the air, able to count the number of dust particles or other particles present in the air, preferably an optical sensor, for example of the “GP2Y1010AU0F Sharp” type or other, designed for detecting the particles in a volume of air, generally the number of particles of a given size or smaller in one cubic metre of air. Here, it makes it possible to display an instantaneous value of the number of particles per cubic metre of air in a range of defined values of size, fixed in the example at 2.5 micrometres and smaller (PM 2.5). These particles are in fact particularly harmful to human health, going deep into the respiratory tree, or even into the bloodstream after passing through the wall of the pulmonary alveoli.

Here, the sensor is arranged on the windscreen 5 of the vehicle, unobtrusively in a corner thereof, being made integral therewith by a suitable fixing means, preferably detachable, for example by contact jamming.

The device may comprise several sensors of this type for providing more precise information on air quality, for example two sensors each arranged in a corner of the windscreen, each capturing particles of different sizes, notably 2.5 micrometres and smaller and 10 micrometres and smaller per cubic metre of air.

The device may also comprise a sensor as specified above of the density of NOx gases in the air, for example replacing the aforementioned sensor of 10 micrometres and smaller per cubic metre of air.

The device also comprises a means for displaying and/or recording 7 said number of particles correlated to a particular measurement location of the sensor, the device moreover being configured to be mounted and to operate onboard a vehicle, here a motor vehicle. In the example, this means comprises a box 9 mounted on the dashboard 11 of the vehicle, connected to a GPS (Global Positioning System) device 13 and to the sensor 3. The box is configured for displaying and/or recording the data from the GPS device and the data from the sensor at each measured point.

Preferably acquisition of the data is stored in memory to be recorded subsequently or simultaneously by a, for example wireless, transmitting means 24, mobile or integrated telephone support etc., here by means of a data processing unit such as a portable computer 15 shown in FIG. 3 and a recording or printing unit, for example a printer 17 connected to the computer. A map 18 of the data from the GPS device and of the data from the sensor at each measured point of the measurement circuit 20 is printed on paper. The legends circled on this circuit represent the values of the number of fine particles per cubic metre of air at each measured point.

The information collected may also be recorded on a database 16, used moreover in the form of applications on a portable telephone, tablet, computer, “GSM”, “GPS” etc.

Here, the box 9 comprises a data display screen 19, notably displaying a geographical map of the measurement location (GPS map) supplemented with points of said values of the number of particles reported by the sensor, which is convenient for directly monitoring the development of the measurements.

It is thus possible to determine, continuously or discontinuously during travel of the vehicle, mapping of the density of fine particles in the air of said location.

This discontinuous manner of determining the capture of particles may be established at particular points to be measured in said location, for example sensitive places of a given geographical zone of the location, each point being allocated a measurement of the number of particles, for example the number of fine particles per cubic metre.

Measurement may be effected when the vehicle stops, or while it is moving. Stopping may be delayed for a specified period to allow re-initialization or recalibration of the sensor at each measurement point on a measurement circuit, from the vehicle, in said location.

It is also possible to travel at a moderate speed in the measurement circuit depending on the capacity for re-initialization of the sensor, for example below 30 km/hour, allowing the sensor to perform a measurement “on the fly” (while moving) and with an acceptable margin of error of measurement of the air surrounding the vehicle.

Re-initialization of the sensor makes it possible to provide threshold values and therefore guarantee the measured values according to a margin of error below 0.20, or up to max. 0.40.

Advantageously, said sensor is mounted on the vehicle in a position favourable to good reception of the air to be measured for capturing the particles (and/or the density of NOx gases) in the air of the measurement point. Here, this sensor is mounted on the windscreen, but it may be mounted on an air intake unit of the engine or of the passenger compartment, on the roof or bonnet of the vehicle or a mast or other arm and accessories fixed to the vehicle.

The sensor may thus be mounted on a mast of the vehicle, or a moving, telescopic arm, adjustable for orientation and/or height, notably allowing measurement at variable height, for example according to a line of vertical projection from the measurement point.

Measurement may thus be performed at several heights for a given point; it may also be performed in the surroundings of the given point, at a distance close to the point or to the vertical from the point, for example by orienting the mast and the sensor at a given measurement height. It is thus possible to determine the density of particles (and/or the density of NOx gases in the case of a gas sensor) at push-chair height, at the height of the pedestrians on the pavement, at ground floor level or higher, or even inside a residential building.

The sensor may also be configured to minimize the effect of the movement of air, wind and/or of the vehicle's speed, for example by fitting it with a peripheral protective skirt configured to allow air to pass through while moving, for example an external grille 21 through which the air passes, as shown in FIG. 2.

Furthermore, the sensor may comprise a device for evacuating the residual air from the sensor, for example a device for blowing neutral air into the sensor (not shown), before each new measurement, which may speed up initialization of the sensor. The sensor may advantageously be equipped with an ultrasonic cleaning system, making its use reliable and durable, thus allowing new usage of the sensor, intensive and in mobile conditions.

Said values may also be initialized at certain points of the map, with reference to those corresponding to standardized particle sensors (for accurate measurement), for example fixed point sensors arranged on the circuit, to supplement and monitor the mobile on-board network.

The invention also relates to a method for measuring and displaying and/or recording particles and/or the density of NOx gases in the air by means of at least one sensor 3 of particles and/or of the density of NOx gases in the air, able to count the number of particles of a size between defined values in a volume of air and/or the density of NOx gases, and by means of a device 7, 15, 17 for displaying and/or recording said number and/or density correlated to a defined location, the whole moreover being configured to be mounted and to operate onboard a vehicle 2, said method comprising acquisition of said number and/or density at points of a measurement circuit onboard said vehicle.

The recording of values may be sequenced at a defined duration between two consecutive measurement points, for given forward travel of the vehicle corresponding to a given distance travelled so as to obtain a regular point display of said values of particles and/or density of NOx gases on the vehicle's circuit.

The operation of the device is simple and as accessible as possible. The device 1 is activated from the vehicle's instrument panel. It is a question of maintaining operation on the pre-established measurement circuit by following the development of the measurement displayed for optionally supplementing the circuit as a function of relevance of the values read. Terminal recording is operated permanently by means of the home computer 15.

Many embodiment variants of the invention may be envisaged employing the various features presented above, alone or in combination.

The invention thus offers the possibility of knowing in real time, or even of predicting over time (on a development curve), with direct display and recording of the densities of particles and/or NOx gases in the air of a given geographical zone, notably a sensitive polluted urban zone.

1. A device for capturing and recording fine particles and/or the density of NOx gases in the air, comprising:
a sensor of particles and/or of the density of NOx gases in the air, able to count a number of dust particles or other particles present in the air, of a size between defined values in a volume of air, and/or density of NOx gases, and a means for displaying and/or recording said number and/or density correlated to a particular measurement location of the sensor, the device being configured to be mounted and to function onboard a moving vehicle, in which said sensor may be initialized at certain points of the map, or even recalibrated, with reference to standardized sensors.

2. The device according to claim 1, wherein said displaying and/or recording means comprises a screen for displaying said number and/or density or an acquisition device of said number and/or density on a recording medium.

3. The device according to claim 1, wherein said sensor is an optical dust sensor, for example of the “GP2Y1010AU0F Sharp” type, designed for detecting and counting the particles in the air.

4. The device according to claim 1, wherein said sensor is mounted on the vehicle in a position favourable for receiving the air to be measured for capturing the particles and/or density of NOx gases in the air of the measurement point, being configured to minimize the effect of the movement of air, of the wind and/or of the vehicle's speed.

5. The device according to claim 1, wherein said sensor is equipped with a peripheral protective skirt configured to allow air to pass through while moving, for example provided with a grille through which air passes.

6. The device according to claim 1, comprising a device for evacuating residual air from the sensor, for example a device for blowing neutral air into the sensor, before each new measurement, and a device for cleaning the sensor, for example with ultrasound.

7. The device according to claim 1, wherein said sensor may be mounted on a mast of the vehicle, or even a moving, telescopic arm, adjustable for orientation and/or height.

8. The device according to claim 1, being coupled to a so-called GPS (Global Positioning System) device for combining a localized point display of the density of the particles or nitrogen oxide gases with a GPS geolocation map.

9. The device according to claim 1, wherein said sensor may be mounted on an air intake unit of the engine or of the passenger compartment, on the roof or the bonnet of the vehicle or a mast or other arm and accessories fixed to the vehicle.

10. A method for measuring and displaying and/or recording particles and/or the density of NOx gases in the air by means of at least one sensor of particles and/or of the density of NOx gases in the air, able to count the number of particles of a size between defined values in a volume of air and/or the density of NOx gases, and by means of a device for displaying and/or recording said number and/or density correlated to a defined location, the whole moreover being configured to be mounted and to operate onboard a vehicle, said method comprising acquisition of said number and/or density at points of a measurement circuit onboard said vehicle.
